(19) United States
(12) Patent Application Publication
(10) Pub. No.: US 2022/0258113 A1
(43) Pub. Date: Aug. 18, 2022

(54) AGITATION PLATFORM FOR MAINTAINING HOMOGENEITY OF SOLUTIONS

(71) Applicant: Brammer Bio MA LLC, Cambridge, MA (US)

(72) Inventors: Yih Horng Tan, Cambridge, MA (US); Elizabeth Grafeld, Cambridge, MA (US); Jeffrey S. Anderson, Cambridge, MA (US); Jessica T. Beyer, Cambridge, MA (US)

(21) Appl. No.: 17/733,551

(22) Filed: Apr. 29, 2022

Related U.S. Application Data
(63) Continuation of application No. PCT/US20/58290, filed on Oct. 30, 2020.
(60) Provisional application No. 63/088,782, filed on Oct. 7, 2020.
(60) Provisional application No. 62/928,115, filed on Oct. 30, 2019.

(51) Int. Cl. B01F 31/10; B01F 31/20; B01F 35/513

(52) U.S. Cl. CPC B01F 31/10; B01F 35/513; B01F 31/201

(57) ABSTRACT

According to embodiments of the present disclosure, apparatuses, systems, and methods are provided that enable the maintenance of homogeneity of biomaterials during aseptic fill-finish. In various embodiments, an apparatus for aseptic fill-finish provided herein includes an agitation device. The apparatus further includes a platform coupled to the agitation device. The platform is rotatable about a first axis. The apparatus further includes at least one shaft extending from the platform. The shaft is adjustable in length. The apparatus further includes at least one arm extending from the shaft. The at least one arm has at least one fixation mechanism. The at least one fixation mechanism is configured to receive a container having a sealed compartment containing a biomaterial and maintain the container in a substantially vertical orientation. The platform is configured to engage at least a portion of the container.

### REFERENCE TO RELATED APPLICATIONS

This application claims the benefit of U.S. Provisional Patent Application No. 62/928,115, filed Oct. 30, 2019, and U.S. Provisional Patent Application No. 63/088,782, filed Oct. 7, 2020, the contents of each of which are hereby incorporated by reference in their entirety. This application is a continuation and claims the benefit of Patent Cooperation Treaty Application No. PCT/US20/58290, filed on Oct. 30, 2020, the contents of which are hereby incorporated by reference in their entirety.

### FIELD

The present disclosure relates to apparatuses, systems, and processes for aseptic fill-finish of biomaterials while agitating the biomaterials.

### BACKGROUND

Maintaining homogeneity of solutions or formulations under aseptic conditions is critical during bioprocess and pharmaceutical manufacturing. Aseptic processes are designed to minimize exposure of sterile articles to the potential contamination hazards of the manufacturing operation. Limiting the duration of exposure of sterile product elements, providing the highest possible environmental control, optimizing process flow, and designing equipment to prevent sub-micron airborne contamination of Class 100 (ISO 5) clean areas are essential to assure sterility.

A critical step where handling of a biomaterial, e.g., a drug product, under aseptic conditions, is of utmost importance during bioprocess and pharmaceutical manufacturing is the fill-finish step. Importantly, once a pharmaceutical or biological product is placed in a sealed, aseptic container, additional transfer of product between containers increases the likelihood of product contamination. If the fill-finish process is not sterile, it can result in significant safety risks to the end user of the resulting product. Additional adverse consequences of product contamination include reputational harm, lost revenue, expensive recalls, complicated regulatory issues, and/or costly litigation. Thus, aseptic filling processes are of paramount importance to pharmaceutical and bioprocess manufacturing.

In some fill/finish processes involving pharmaceutical and/or biopharmaceutical products (e.g., gene therapy, immunotherapy), maintaining homogeneity of the product during fill-finish is important in order to achieve uniformity of the product across individual units or vials. Specifically, non-homogeneity of product would result in an unacceptable variation in the amount of active agent from one vial to another. Such variation could result in patients receiving doses of the product that are either too low (potentially rendering the treatment ineffective) or too high (raising the risk of an overdose).

Traditional methods for maintaining homogeneity rely on extraneous components in direct contact with the pharmaceutical and/or biopharmaceutical product to agitate and/or mix the sterilized product. For example, a magnetic stir rod may be placed in the container. The introduction of extraneous components for mixing (e.g., stirring rods, magnetic stirring bar, and/or impellor) into the product itself during the fill/finish process increases the likelihood of contamination or degradation of product due to excessive collision/friction with the structure of the extraneous component. Also, it is common practice in the industry to squeeze and/or massage the bag containing the drug product during fill-finish; however, this poses the risk of puncturing the bag during fill-finish and also it is not a viable solution when scaling up or dealing with large volumes of drug product.

### SUMMARY

The present disclosure provides apparatuses, systems and processes for aseptic fill-finish of solutions (e.g., biomaterials, including drug products) with agitation of the solutions, without the need for any extraneous components (e.g., stir bars, rods, or impellers). Thus, in some embodiments, the apparatuses, systems and processes described herein are particularly useful for maintaining homogeneity of solutions during processing, such as aseptic fill-finish.

Embodiments of the present disclosure generally relate to an agitation platform for mixing or agitating solutions, including biomaterials, e.g., drug products. In a particular embodiment, the present disclosure describes an agitation platform for agitating and/or mixing sterile biological materials, e.g., drug products, contained within sealed containers during fill-finish processes, without the need to introduce any extraneous components in contact with the sterile material/product.

In some embodiments, an apparatus for aseptic fill-finish while agitating is provided. The apparatus includes an agitation device and a platform coupled to the agitation device. The platform is rotatable about a first axis. The apparatus further includes at least one shaft extending from the platform. In some embodiments, the shaft is adjustable in length. The apparatus further includes at least one arm extending from the shaft and the at least one arm has at least one fixation mechanism. The at least one fixation mechanism is configured to receive a container having a sealed compartment containing a biomaterial and maintain the container in a substantially vertical orientation. The platform is configured to engage at least a portion of the container.

In some embodiments, the apparatus includes at least one shaft extending perpendicularly from a midpoint of the platform and at least one arm extends perpendicularly from the shaft, and the at least one fixation mechanism extends perpendicularly from the at least one arm. In some embodiments, the at least one shaft includes a first shaft disposed proximate a first edge of platform and a second shaft disposed a second edge of the platform. In some embodiments, the first shaft is aligned with the second shaft.

In some embodiments, the platform comprises a planar surface. In other embodiments, the platform comprises a non-planar surface. In some embodiments, the platform is configured to rotate about a second axis. In certain embodiments, the platform is configured to rotate about first and second axes simultaneously. In some embodiments, the platform is configured to move in a plane defined by the first axis and the second axis. In yet other embodiments, the platform is configured to rotate in first, second and third axes.

In some embodiments, the container is a flexible bag. In some embodiments, the container is made of a polymer. In certain embodiments, the container comprises a biomaterial. Examples of biomaterials include, include but are not limited to, pharmaceutical agents, recombinant vectors (e.g., for use in gene and/or cell therapies), antibodies (e.g., monoclonal antibodies), therapeutic proteins (e.g., Fc fusion proteins), inhibitory nucleic acid molecules (e.g., inhibitory RNA molecules), and genetically-modified cells (e.g., CAR-T cells).

In various embodiments, the apparatus described herein is not fluidly coupled to a pump, is not coupled to a second container and/or does not include a stirring mechanism.

In certain aspects, provided herein are methods for aseptic fill-finish while agitating. In some embodiments, a method for fill-finish includes dispensing biomaterials into a container (e.g., a container provided herein). In some embodiments, the container has a first end and a second end. In certain embodiments, the method includes affixing the first end of the container to an agitation device (e.g., an agitation device described herein). For example, in some embodiments, the agitation device has a platform rotatable about a first axis, at least one shaft extending from the platform, and at least one arm extending from the shaft. The first end of the container is releasably affixed to the at least one arm. In some embodiments, the method includes positioning the container in a substantially vertical orientation. In certain embodiments, the method includes engaging at least a portion of the second end of the container with the platform. In some embodiments, the method includes rotating the platform about the first axis. Examples of biomaterials include, but are not limited to, pharmaceutical agents, recombinant vectors (e.g., for use in gene and/or cell therapies), antibodies (e.g., monoclonal antibodies), therapeutic proteins (e.g., Fc fusion proteins), inhibitory nucleic acid molecules (e.g., inhibitory RNA molecules), and genetically-modified cells (e.g., CAR-T cells).

In some embodiments, the method further comprises the step of processing the biomaterials prior to dispensing. In some embodiments, the step of processing comprises subjecting the biomaterials to a filtration step prior to dispensing the biomaterials into the container.

In various embodiments, the platform comprises a planar surface or a non-planar surface.

In some embodiments, affixing the first end of the container includes adjusting a length of the shaft. In some embodiments, rotating includes displacement of the platform about a second axis. In certain embodiments, rotating includes displacement of the platform in a plane defined by the first axis and/or a perpendicular second axis. In some embodiments, rotating includes displacement of the platform about the first and second axes simultaneously.

### BRIEF DESCRIPTION OF THE DRAWINGS

FIG. 1 illustrates an exemplary system for aseptic fill-finish processing which employs a pump to move a sample from one container to a second container.

FIG. 2A illustrates an exemplary system for aseptic fill-finish processing which employs a recirculation loop. FIG. 2B illustrates an expanded view of a recirculation loop portion of a system for aseptic fill-finish processing.

FIG. 3 illustrates an exemplary system for aseptic fill-finish processing with an agitation device.

FIGS. 4A-4B illustrate exemplary agitation apparatuses for aseptic fill-finish processes according to embodiments of the present disclosure.

FIG. 5 illustrates an exemplary system for aseptic fill-finish processing with an agitation apparatus according to embodiments of the present disclosure.

FIG. 6 illustrates an exemplary agitation apparatus for aseptic fill-finish processes according to embodiments of the present disclosure.

FIG. 7 illustrates an exemplary agitation apparatus for aseptic fill-finish processes according to embodiments of the present disclosure.

FIG. 8 illustrates a method for fill-finish processing with an agitation platform according to embodiments of the present disclosure.

FIG. 9 shows the vector genome (Vg) titer (which is a measure of viral quantification) for vials taken during the final fill for Example E.

FIG. 10 shows the osmolality results for vials taken during the final fill for Example E.

### DETAILED DESCRIPTION

Certain drugs and/or medical devices (including single-use systems) may be sterilized using terminal sterilization, such as, for example, autoclave, irradiation, ethylene oxide, or decontaminated using vaporized hydrogen peroxide (VHP). Terminal sterilization usually involves carefully filling and sealing product containers under environmental conditions, which minimize the microbial and particulate content of the in-process product and to help ensure that the subsequent sterilization process is successful. In most cases, the product, container, and closure have low bioburden, but they are not sterile. The product in its final container is then subjected to a sterilization process such as moist heat or irradiation. Unlike terminally-sterilized filled drugs and/or medical devices, the stability of aseptically-filled drugs and/or biological products may be affected by traditional terminal sterilization techniques, such as, for example, steam autoclave, dry heat ovens, ethylene oxide, and irradiation (e.g., Cobalt 60 Gamma or E Beam).

Many global regulatory requirements exist for aseptic/sterile fill/finish manufacturing. In the United States, for example, these regulatory requirements may be found in the FDA September 2004 Guidance for Industry (“FDA Guidance”). This FDA Guidance describes that in an aseptic process, the drug product, container, and closure may first subjected to sterilization methods separately, as appropriate, and then brought together. Because there is no process to sterilize the product in its final container, it is critical that containers be filled and sealed in an environment, which minimizes the microbial and particulate content of the product. Aseptic processing involves more variables than terminal sterilization. Before aseptic assembly into a final product, the individual parts of the final product are generally subjected to various sterilization processes. For example, glass containers are subjected to dry heat depyrogenation; rubber closures are subjected to moist heat; and liquid dosage forms are subjected to sterile filtration. Each of these sterilization processes requires validation and control. Each process could introduce an error that ultimately could lead to the distribution of a contaminated product. Any manual or mechanical manipulation of the sterilized drug, components, containers, or closures prior to or during aseptic assembly poses the risk of contamination and thus necessitates careful control. A terminally sterilized drug product, on the other hand, undergoes final sterilization in a sealed container, thus limiting the possibility of error.

The FDA Guidance further states that sterile drug manufacturers should have a keen awareness of the public health implications of distributing a nonsterile product. Poor CGMP conditions at a manufacturing facility can ultimately pose a life-threatening health risk to a patient.

In the U.S., certain rules were written to codify sterile processing. For example, 21 CFR 211.113 (b) states that appropriate written procedures, designed to prevent microbiological contamination of drug products purporting to be sterile, shall be established and followed. Such procedures shall include validation of all aseptic and sterilization processes. Another section, 21 CFR 211.167 (a) states that for each batch of drug product purporting to be sterile and/or pyrogen-free, there shall be appropriate laboratory testing to determine conformance to such requirements. The test procedures shall be in writing and shall be followed.

In some aseptic fill/finish processes involving pharmaceutical and/or biopharmaceutical products (e.g., gene therapy, immunotherapy), the product may require stirring or agitation during the fill/finish process to ensure product uniformity across many individual units. Because introducing any external components (e.g., stirring rods, magnetic stirring bar, and/or impellor) into the product itself during the fill/finish process may increase the likelihood of product aggregation or contamination, and damage due to excessive collision/friction with the structure of the external component, it is desirable to agitate such products in the respective aseptic containers without exposure to external components.

In addition to minimizing the risk of contamination during fill finish, ensuring homogeneity of a drug product during fill-finish is also desirable so that each vial receives a uniform drug product. However, achieving product homogeneity can be challenging as it is not desirable to introduce any extraneous components for mixing or agitating the drug product. The present disclosure provides apparatuses, systems, and processes for ensuring homogeneity of the drug product during aseptic fill-finish.

Without wishing to be bound by theory, it is contemplated that the apparatuses, systems, and methods described herein can also be used for many other applications, for example, mixing or agitation of any solution under aseptic conditions without the need to introduce any external components (e.g., stir bars, rods, impellors, etc.). In various embodiments, the apparatuses, systems, and methods described herein may be used for mixing and/or agitation of a biomaterial during the fill-finish step. In other embodiments, the apparatuses, systems, and methods described herein may be used for mixing and/or agitation of a biomaterial prior to the fill-finish step, where mixing under aseptic conditions is desirable.

In various embodiments, apparatus described herein includes a container. In some embodiments, the container is a sealable bag. In various embodiments, the bag may have one or more ports, and corresponding valves, for transferring a pharmaceutical and/or biopharmaceutical liquid into or out of the container. It is contemplated that any commercially available flexible container may be used in the embodiments described herein. In various embodiments, the container may be made of a polymer. Examples include an OctoPlus® or Mobius® bag.

In various embodiments, agitation may be provided to the container via an agitation device operably coupled to a base/platform. The terms “agitation device,” “agitation apparatus,” and “agitation platform” may be used interchangeably through the disclosure. In various embodiments, the agitation device is a rocker. In various embodiments, the agitation device may affect movement of the base within a plane defined by x and y axes. In various embodiments, the base may be substantially planar (e.g., flat). In various embodiments, the base may be non-planar (e.g., curved). In various embodiments a curved base may reduce motion of the container caused by sloshing liquid (e.g., pharmaceutical/biopharmaceutical) as the liquid is mixed or agitated by the agitation device. In some embodiments, the agitation device (e.g., a rocker) may rotate in one axis, or two axes or three axes. Any suitable commercially available rocker may be used in various embodiments described herein.

In various embodiments, agitation of a pharmaceutical and/or biopharmaceutical product by an agitation device during aseptic fill/finish processing promotes drug product homogeneity following downstream filtration and during the aseptic filling process. In various embodiments, agitation may include see-saw rocking, tilting, gyratory (i.e., combined motions of an orbital shaker and a rocker), and/or orbital mixing (e.g., 3D mixing, nutation) (in orbits/min). In various embodiments, control of an agitation device may be implemented by a fixed tilt angle and/or via multiple shaking modes (e.g., continuous, periodic/pulsed, gradual, abrupt, etc.).

Because the approaches described herein obviate the need to introduce any additional components (e.g., stirring mechanisms, such as rods, stir bars, or impellers) that potentially generate particulates, degrade biomaterials, cause product aggregation, or entrain air into the product, the approaches described herein significantly reduce the likelihood of product contamination during the fill-finish step or any other step where an apparatus described herein may be employed during bioprocess manufacturing. In various embodiments, the approaches described herein enhance smooth movement of product within its sterile container without generating splashing or stratifying material. In various embodiments, the approaches described herein allow for low foaming agitation and/or gentle continuous agitating or stirring of drug product with variable speed, e.g., from approximately 5 rpm to approximately 60 rpm, or variable speed and tilt angle or 3D mixing.

In various embodiments, countercurrent or eddy motion displacing the pharmaceutical and/or biopharmaceutical product from one end of a container to the other opposite end may promote mixing or agitation with or without an agitation device.

In various embodiments, prior to filling a container with a pharmaceutical and/or biopharmaceutical product solution, filtration may be used to sterilize the pharmaceutical and/or biopharmaceutical product solution. In various embodiments, as described in FDA guidelines regarding filtering of pharmaceutical and/or biopharmaceutical products, such filters usually have a rated pore size of approximately 0.2 μm or smaller. Other suitable filter sizes may be used as is known in the art. In various embodiments, redundant sterilizing filters may be used. Whatever filter or combination of filters is used, validation may include microbiological challenges to simulate worst-case production conditions for the material to be filtered and integrity test results of the filters used for the study. Product bioburden may be evaluated when selecting a suitable challenge microorganism to assess which microorganism represents the worst-case challenge to the filter. The microorganism Brevundimonas diminuta (ATCC 19146) when properly grown, harvested and used, may be a common challenge microorganism for 0.2 μm rated filters because of its small size (0.3 μm mean diameter). The manufacturing process controls may be designed to minimize the bioburden of the unfiltered product. Bioburden of unsterilized bulk solutions may be determined to trend the characteristics of potentially contaminating organisms.

In various embodiments described herein, the terms “mixing” and “agitating” are used interchangeably to describe a method to ensure uniformity or homogeneity of a solution (e.g., a biomaterial). A homogenous solution can be any liquid that has the same proportions of its components throughout any given sample of the liquid. In various embodiments, a homogenous solution is a solution containing a drug product. Mixing or agitating a solution, as described herein, may be used for stirring the contents of a mixture, such that any solids in the mixture become fully dissolved thereby resulting in a homogenous liquid or solution. Mixing or agitating a solution as described herein, may also be used in the context of a fill-finish step of a bioprocess manufacturing process, such that the drug product (e.g., a biologic such as a gene therapy vector or a monoclonal antibody) being fill-finished has a homogenous titer across different vials that are being filled.

In some fill finish processes, one or more pumps (e.g., a peristaltic pump) are used to pump the solution from a first container (e.g., a bag) to a second container (e.g., a bag), thereby altering current flow and agitating and/or mixing the solution via small current flow changes in the two containers. In some instances, use of a pump may generate non-consistent current flow over the entire transfer process and may require stopping and starting the pump(s) to control flow rate or filling rate. Moreover, mixing may not occur when the biomaterial is transferred from the first bag to the second bag.

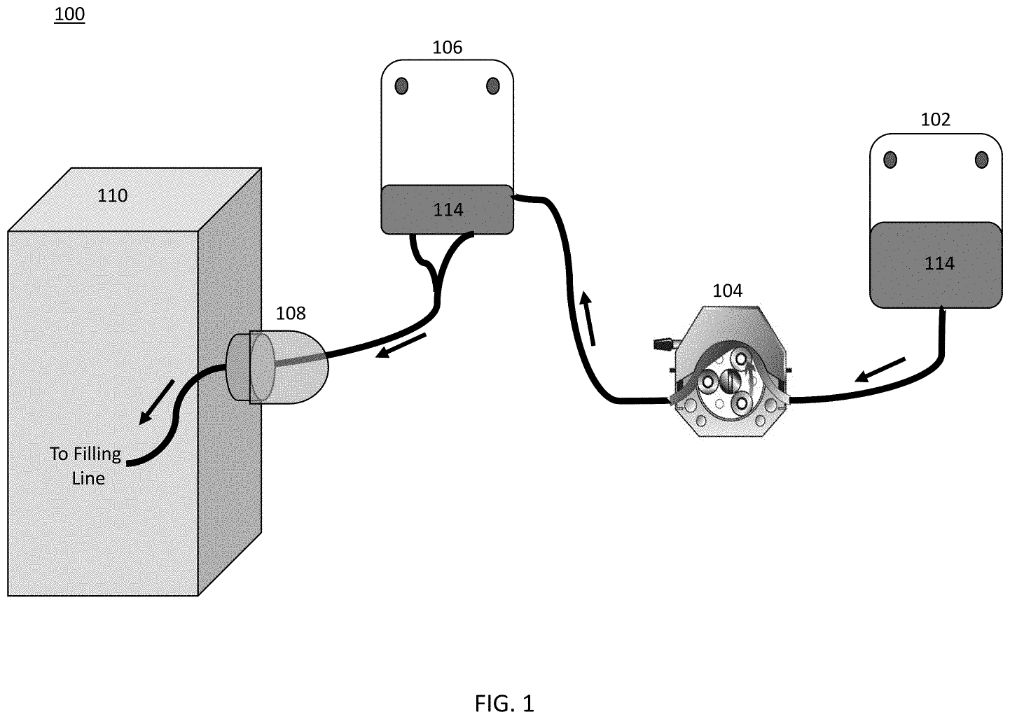

FIG. 1 illustrates a system 100 for aseptic fill-finish processing. In FIG. 1, a container 102 (e.g., a Mobius® bag) may be connected to a pump 104 (e.g., peristaltic pump) via sterile tubing (e.g., silicon tubing of suitable diameter). In various embodiments, the container 102 includes a pharmaceutical and/or biopharmaceutical product ready for fill-finish, which can be maintained at room temperature. In various embodiments, the container 102 may have any suitable capacity (e.g., 0.3 L, 1 L, 5 L, 8 L, 10 L, 20 L). In various embodiments, any of the components of the system 100 may be sterilized using a decontamination technique described above prior to coming into contact with the pharmaceutical/biopharmaceutical product. The system 100 further includes a second container 106 (e.g., OctoPlus® bag) connected to the pump 104 via tubing. The first container 102 and/or the second container 106 may include the pharmaceutical and/or biopharmaceutical product 114. In various embodiments, the second container 106 may have any suitable capacity (e.g., 0.3 L, 1 L, 5 L, 8 L, 10 L, 20 L). In various embodiments, the second container 106 may be smaller than the first container 102. In various embodiments, the second container 106 may be equal in size to the first container 102. In an exemplary embodiment, the second container 106 is filled to a predetermined limit, e.g. approximately 5 L (out of a maximum capacity of 8 L, for example). In various embodiments, each container 102, 106 may have a maximum fill capacity that is less than the maximum capacity of the container to reduce the risk that the container could fail (e.g., burst). The second container 106 is connected to a rapid transfer port (RTP) and filler assembly 108. The RTP and filler assembly 108 is connected to an isolator 110 (e.g., any suitable commercial isolator, such as a SKAN isolator) for aseptic filling of individual product units. In various embodiments, the isolator may include a hydrogen peroxide decontamination system, various air filtration units (e.g., HEPA filters), pressure regulation capabilities, a filtered exhaust for aseptic filling, and/or a filling needle of a predetermined size as is known in the art (e.g., 3.2 mm). In various embodiment, the filling parameters may include a predetermined fill volume per vial (e.g., 0.1 ml, 0.2 ml, 0.3 ml, 0.4 ml, 0.5 ml, 1 ml, 2.5 ml, 5 ml, 7.5 ml, 10 ml, 20 ml, 30 ml, 40 ml, 50 ml, 60 ml, 70 ml, 80 ml, 90 ml, 100 ml, etc.), temperature control, and/or filling duration (e.g., 5 min, 10 min, 30 min, 1 hrs, 5 hrs, 10 hrs, etc.). In various embodiments, the predetermined fill volume may be between 0.1 ml to 100 ml based on target fill weight. In various embodiments, the fill duration may be dependent on batch size. In various embodiments, the filling process may be performed in a sterile environment defined by good manufacturing practices (e.g., Grade A, Grade B, etc.).

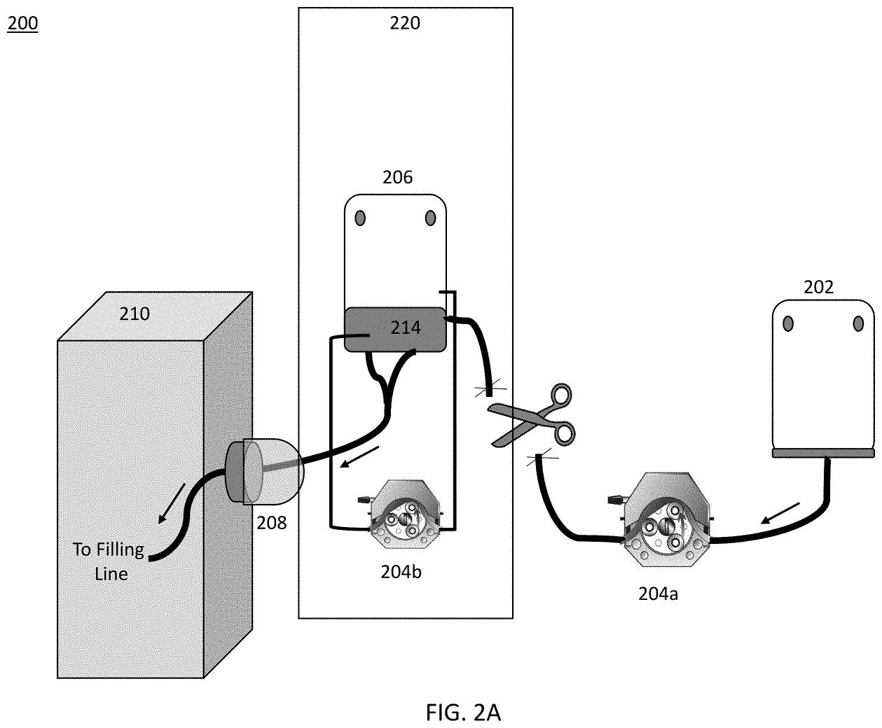

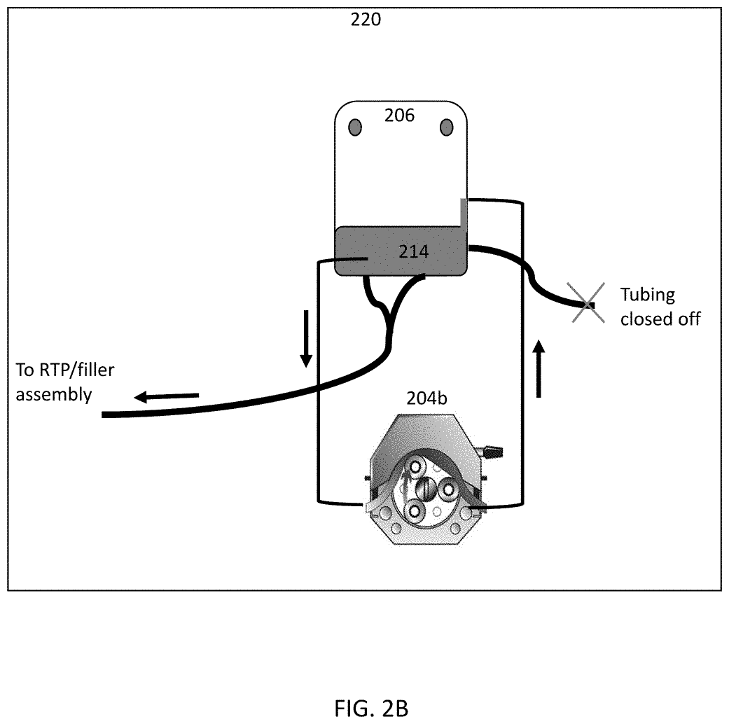

FIG. 2A illustrates a system 200 for aseptic fill-finish processing, which employs a recirculation pump. In the system 200, recirculation of the pharmaceutical/biopharmaceutical product 214 may be performed after the product is transferred from the first container 202 (e.g., Mobius® bag) via tubing to a first pump 204a and into a recirculation loop portion 220. The recirculation loop portion 220 includes a second container 206 connected to a second pump 204b in a recirculation arrangement. The second container 206 is also connected to the RTP and filler assembly 208. In various embodiments, the tubing connecting the first pump 204a and the second container 206 may be closed after substantially all of the product 214 has been pumped out of the first container 202 and into the second container 206. In various embodiments, the recirculation loop portion 220 may recirculate the product to thereby mix or agitate the product 214 within the second container 206. In various embodiments, recirculation may occur before any product 214 is transferred to the RTP and filler assembly 208. In various embodiments, recirculation may occur during the transfer of product 214 to the RTP and filler assembly. Similar to the above embodiments, the product is then transferred to an isolator 210.

In various embodiments, the recirculation loop portion 220 connecting the second container 206 with the second pump 204b may be controlled/regulated to be opened and/or closed via one or more valves. When closed, no recirculation may occur and the system 200 may operate in substantially the same way as the system 100 shown in FIG. 1.

In various embodiments, after the pharmaceutical and/or biopharmaceutical product 214 is transferred from the first container 202, the recirculation loop portion 220 may be disconnected or closed-off from the first container 202 via, for example, one or more shut-off valves.

FIG. 2B illustrates an expanded view of a recirculation loop portion 220 of a system for fill-finish processing.

In various embodiments, an agitation device may be incorporated into the aseptic fill-finish system to further improve the mixing and/or agitation capabilities of the system. The first container and/or second container may be placed directly on the base/platform of the agitation device to ensure proper balancing and load distribution when position thereon.

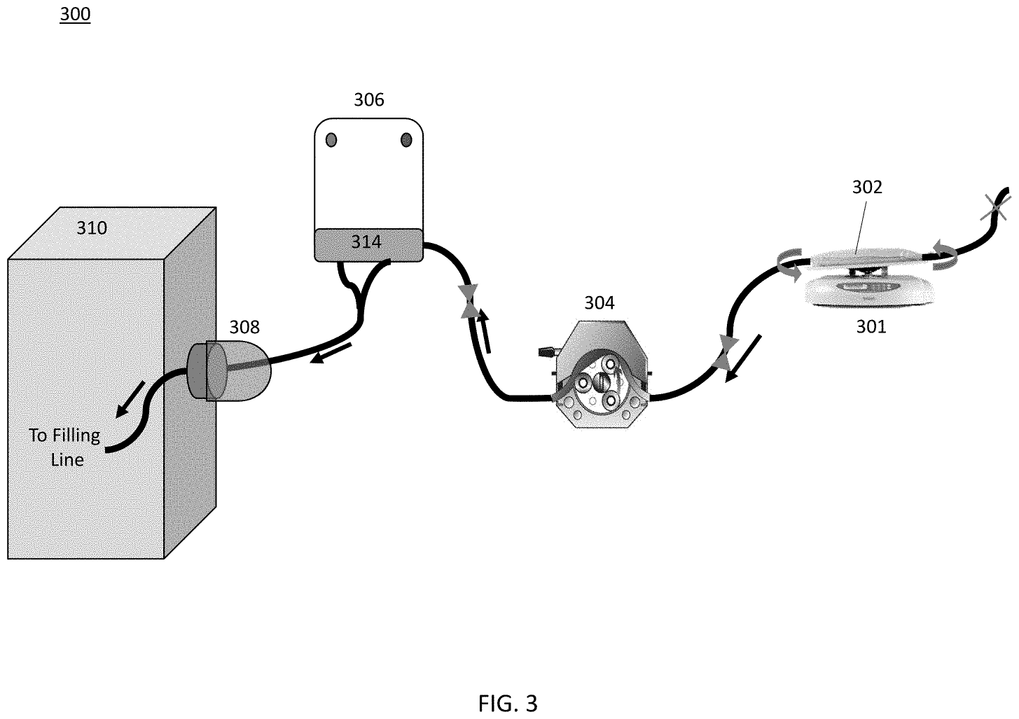

FIG. 3 illustrates a system 300 for aseptic fill-finish processing with an agitation device. The system 300 of FIG. 3 is similar to the system of FIG. 1 but includes an agitation device 301 (e.g., rocker) onto which a first container 302 may be directly placed. In various embodiments, the container 302 may be a bag (e.g., surge bag). The agitation device 301 may agitate the product 314 of the first container 302 before and/or during a transfer of the product 314 to the second container 306 (e.g., OctoPlus® bag, Mobius® bag, etc.) via a pump 304. Similar to the system of FIG. 1, the product 314 is transferred to the second container 306 via the pump 304 followed by transfer to the RTP and filler assembly 308 and the isolator 310, at which point the product 314 are transferred into individual product units (e.g., vials). The system 300 shown in FIG. 3 may allow for continuous agitation and/or mixing and transfer of a pharmaceutical/biopharmaceutical product 314 to the second container 306. In some embodiments, agitation and transfer operations can occur as discrete, stepwise operations performed in sequence such that the product 314 is positioned within a container on the agitation device 301, agitated, and thereafter transferred to the second bag 306; thereafter the process repeats. Alternatively, the agitation and transfer operations can be performed in a temporally overlapping fashion (e.g., simultaneously).

In various embodiments, when a pliable container (e.g., surge bag) is placed directly on an agitation device with a solution contained therein, the solution may flatten out the pliable container and dampen the forces from the agitation device, thus resulting in less effective agitation and/or mixing of the pharmaceutical and/or biopharmaceutical product solution. In various embodiments, structures may be added to the agitation device to support to the container in a particular orientation thus allowing for improved transfer of agitation forces from the agitation device to the solution.

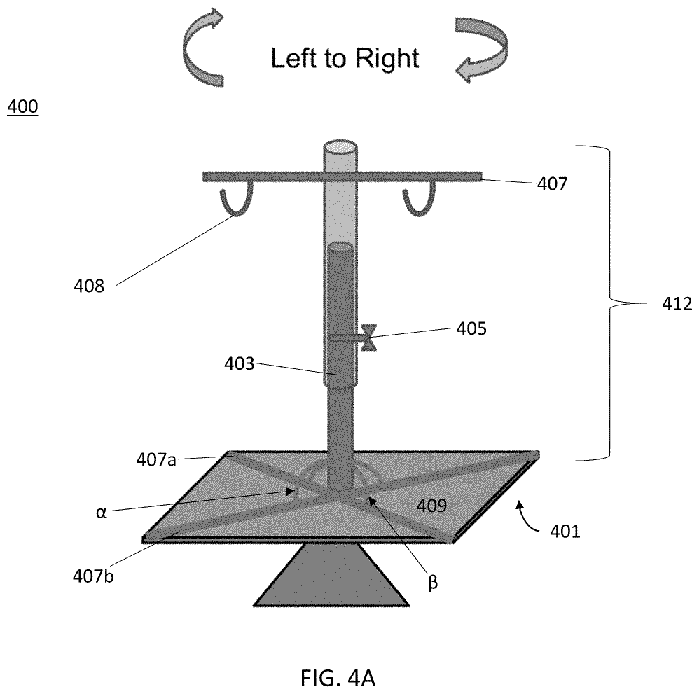

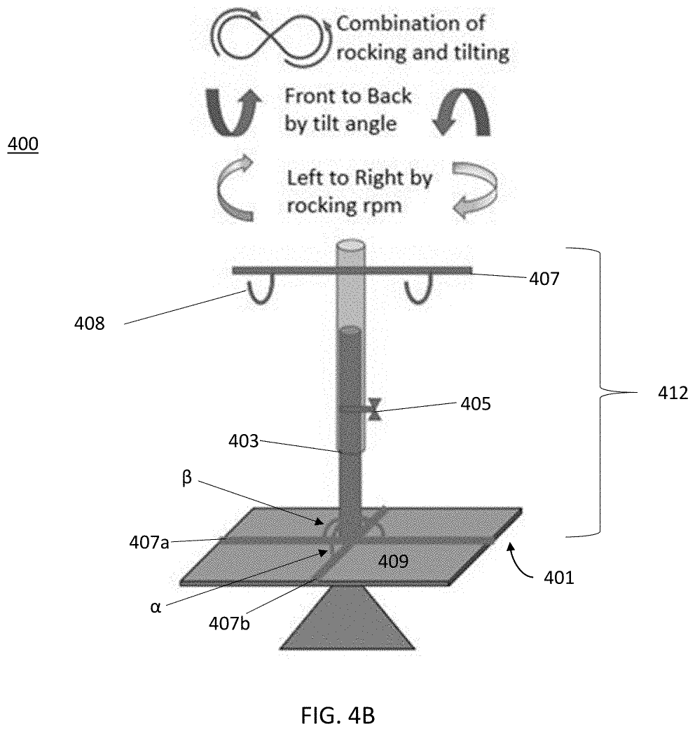

FIGS. 4A-4B illustrate exemplary agitation apparatuses 400 for aseptic fill-finish processes according to embodiments of the present disclosure, which is an improvement over the embodiments described in FIGS. 1-3. FIG. 4A illustrates an agitation device 401 configured to rock left to right in a plane defined by first axis 407a and second axis 407b (as shown by arcs “α” and “β”). The agitation device 401 can include a motor having an attachment member, e.g., fulcrum, to connect to the base 409 and impart motion thereto. The agitation device 401 can operate over a range of speeds and modes (e.g. continuous, periodically pulsed). In some embodiment, the agitation device 401 can operate with fine tune control (e.g., approximately 1 rpm increment adjustment) over the range of speeds, such as approximately 5 rpm to approximately 50 rpm. Additionally, the range of motion of the device (e.g., tilt angle) can range from approximately 1 degrees to approximately 20 degrees, with a 0.1 degree or more increment adjustment control. In some embodiments the agitation and/or mixing is monitored (e.g. detecting formation of bubbles, displacement of bag contents along interior sidewalls, etc.) to ensure the mixing or agitation is within acceptable levels, with the mode/speed of motion of the agitation apparatus adjusted accordingly to stay within acceptable limits.

The agitation device 401 further includes a stand 412 that has a shaft 403 extending substantially in a vertical direction away from the base 409 of the agitation device 401. The shaft 403 may be affixed to the agitation device 401 using any suitable manner as is known in the art, such as, for example, by welding, screws, and/or adhesives. In various embodiments, the shaft 403 may be adjustable in length/height via an adjustment mechanism 405. In some embodiments, the shaft 403 is located at the center of the platform and extends perpendicularly therefrom. In various embodiments, the adjustment mechanism 405 may include a pin or screw.

In various embodiments, the shaft 403 may include a first (lower) component and a second (lower) component. In various embodiments, the components may be cylindrical. In various embodiments, the first (lower) cylindrical component has a smaller outer radius than the inner radius of the second (upper) cylindrical component such that the first cylindrical component is telescopically received within the second cylindrical component. In various embodiments, the first (lower) cylindrical component has a larger inner radius than the outer radius of the second (upper) cylindrical component such that the second cylindrical component is telescopically received within the first cylindrical component (not shown).

In various embodiments, the adjustment mechanism 405 includes a spring-loaded pin on the first component and a plurality of corresponding holes for receiving the pin arranged in a vertical column on the second component. In various embodiments, the shaft further includes one or more arms 407 extending laterally from the shaft 403. In the exemplary embodiment shown, the arms 407 extend bilaterally, and perpendicularly, from the shaft 403, though artisans of ordinary skill will understand alternative configurations are within the scope of the present disclosure. Fixation mechanisms 408 are included along the arms 407 for releasably affixing a container. In various embodiments, the fixation mechanism may include, e.g., hooks, pins, clasps, clamps, and/or magnetic fasteners. In the exemplary embodiments shown, the fixation mechanisms 408 extend downwardly from the arms 407 and are evenly spaced from the shaft 403, though alternative numbers/locations (e.g., upwardly extending, clustering, etc.) of fixation mechanisms 408 can be employed, if desired.

In operation, the height of the shaft 403 is adjusted according to the size of the bag to be placed on the base 409 so as to ensure sufficient contact between the container and surface of the base to efficiently impart momentum into the contents of the container during agitation, or motion, of the base 409. In some embodiments, the height of shaft 403 can be extended so that the container remains fully suspended above, or spaced from, the upper surface of the platform.

FIG. 4B illustrates a similar agitation apparatus 400 as shown in FIG. 4A having an agitation device 401 and a stand having a shaft 403 extending from a base 408, adjustment mechanism 405, and arm 407. In FIG. 4B, the agitation device 401 includes axis 407a and axis 407b. In various embodiments, the agitation device 401 may be configured to move left/right, and or front/back in a plane defined by axis 407a, 407b such that the base 409 shifts or translates within the plane defined by the axes 407a, 407b. Additionally or alternatively, the base 409 can tilt and rotate about one or both axes 407a, 407b (as shown by arcs “α” and “β”). The combination of tilting and rocking may, in some embodiments, provide for better mixing or agitating of product inside a container affixed to the agitation device 401. In some embodiments the mode of movement (translation vs. rotation) can be performed as discrete steps; in some embodiments the mode of movement is performed simultaneously. The base 409 can be configured as a substantially planar surface to receive the bag. In some embodiments, the base 409 can include non-planar surface features to increase the surface area of the platform in contact with the bag, to thereby impart greater energy transfer and momentum into the bag to facilitate mixing or agitation.

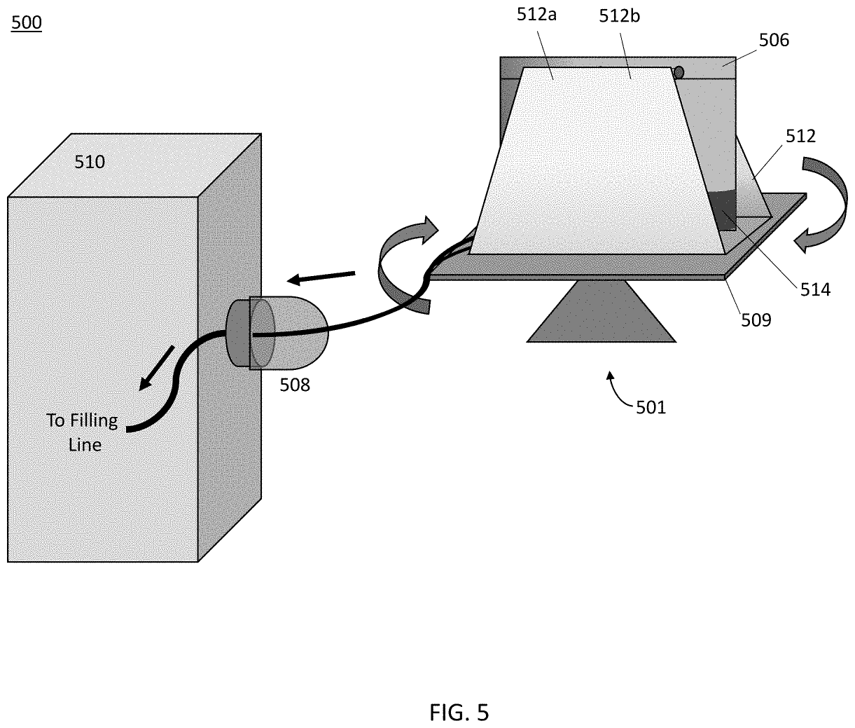

FIG. 5 illustrates a system 500 for aseptic fill-finish processing with an agitation apparatus according to embodiments of the present disclosure, which is an improvement over the embodiments described in FIGS. 1-3. In FIG. 5, an agitation device 501 is provided with a stand 512 for releasably affixing a container 506 (e.g., OctoPlus® bag) thereto. The stand 512 depicted in this exemplary embodiment includes a base 509 and upstanding trapezoidal sidewalls which receive a container 506 therebetween. One skilled in the art will recognize that the sidewalls may include any suitable shape, such as, for example, rectangular or triangular. In various embodiments, the two sidewalls include one or more support members 512a, 512b disposed therebetween, which are configured to support the container 506. The sloped angle of the stand sidewalls allows for easy access to the edges of the container 506. In various embodiments, the agitation device 501 may provide gentle shifting, tilting and/or rotating to the container such that the product 514 may slosh back and forth to thereby agitate the drug product during filling. In various embodiments, any components of the agitation device 501 may be made of a polymer, such as, for example, PET, PU, PE, PVC, polycarbonate and/or PMMA. As the container 506 is agitated by the agitation device 501, the product 514 of the container 506 may be transferred (e.g., drained or pumped) into the RTP and filler assembly 508 followed by processing in the isolator 510.

In various embodiments, as shown in FIG. 5, the system 500 does not include a pump, such as the peristatic pump(s) shown in FIGS. 1, 2A, 2B, and 3. In various embodiments, as shown in FIG. 5, the system 500 does not include a second container into which a pharmaceutical and/or biopharmaceutical product is transferred. In various embodiments, the systems as described herein do not include any stirring mechanisms (e.g., magnetic stir bar) placed in direct contact with the pharmaceutical and/or biopharmaceutical product as any equipment that directly contacts the product after sterilization may contaminate and/or damage the product.

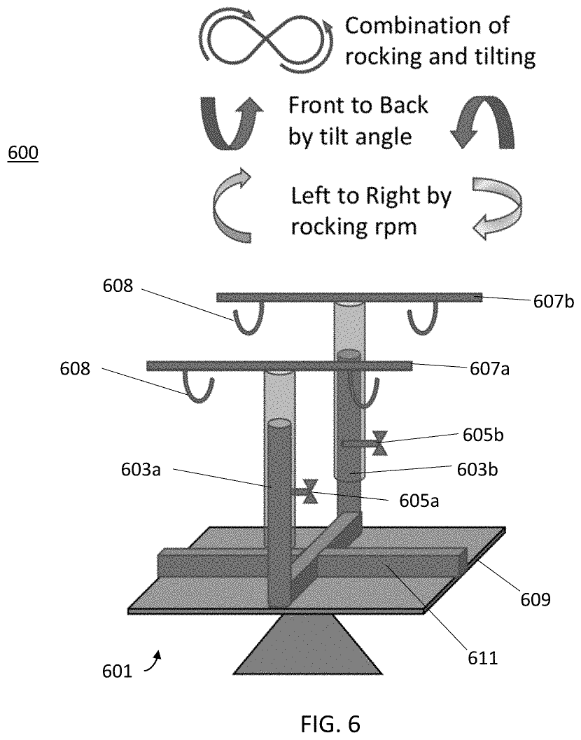

FIG. 6 illustrates an agitation apparatus 600 for aseptic fill-finish processes according to embodiments of the present disclosure, which is an improvement over the embodiments described in FIGS. 1-3. FIG. 6 illustrates an agitation device 601 similar to the agitation devices described above. The agitation device 601 includes two shafts 603a, 603b located proximate opposing edges of a base 609 and aligned along the x-axis (or y-axis) and extending in a substantially perpendicular direction upward from the base 609. In various embodiments, each shaft 603a, 603b may include a respective adjustment mechanism 605a, 605b similar to the adjustment mechanisms described above. The agitation apparatus 600 further includes arms 607a, 607b on the respective shafts 603a, 603b. In various embodiments, the base 609 may include one or more stabilizing beam 611 to provide additional stabilization to shafts 603a, 603b. The agitation apparatus 600 is configured to allow a container (e.g., bag) to be fastened at a first end on the first arm 607a and at a second end on the second arm 607b. In various embodiments, the container is positioned at least partially in contact with the surface of the base 609. In various embodiments, a first end of the container can be attached to the fixation mechanisms 608 of the first arm 607, and a second end of the container can be attached to the fixation mechanisms of the second arm 607b, with a middle portion of the bag draped downwardly in a sling or hammock configuration which engages the base 609.

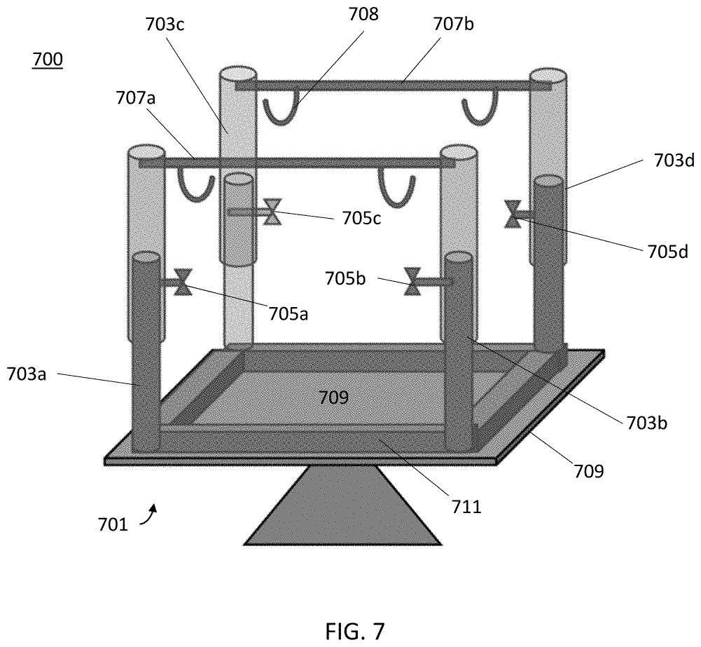

FIG. 7 illustrates an agitation apparatus 700 for aseptic fill-finish processes according to embodiments of the present disclosure, which is an improvement over the embodiments described in FIGS. 1-3. FIG. 7 illustrates an agitation device 701 similar to the agitation devices described above. The agitation device 701 further includes four shafts 703a, 703b, 703c, 703d located proximate the corners of a base 709 and extending in a substantially perpendicular direction upward from the base 709. In various embodiments, the base 709 may include one or more stabilizing beam 711 to provide additional stabilization to shafts 703a-703d. In various embodiments, each shaft 703a-703d may include a respective adjustment mechanism 705a, 705b, 705c, 705d similar to the adjustment mechanisms described above. The agitation apparatus 700 further includes arms 707a connected between shafts 703a, 703b and arm 707b connected between shafts 703c, 703d. The agitation apparatus 700 is configured to allow a container (e.g., bag) to be fastened at a first end on the first arm 707a and at a second end on the second arm 707b. In various embodiments, the container contacts the base 709.

In various embodiments, a feedback system may be implemented such that if filling operations are performing differently from a predetermined standard, the systems described herein may stop transfer (e.g., pumping) of the pharmaceutical and/or biopharmaceutical product solution to the RTP and filling assembly. In various embodiments, the system may optionally, or additionally, stop any agitation devices as described above from agitating and/or mixing the pharmaceutical and/or biopharmaceutical product solution. For example, if a fill-by-weight process is implemented at the filling assembly, and one or more incorrect weights are recorded for one or more vials (e.g., when excessive agitation induces undesirable bubbles in the product), the system may shut off any pumping of pharmaceutical and/or biopharmaceutical product solution and/or operation of any agitation devices.

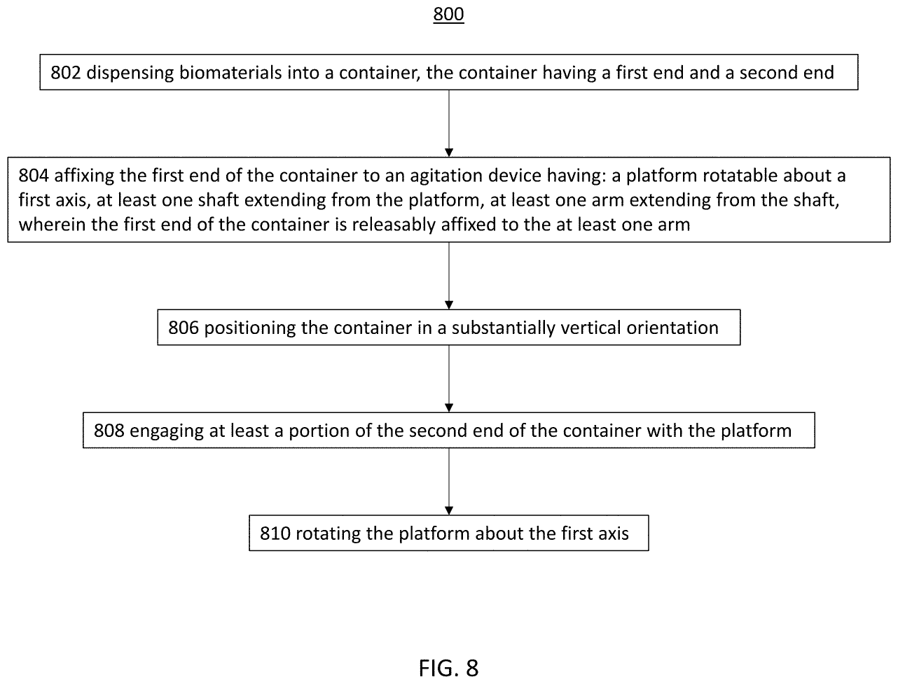

FIG. 8 illustrates a method 800 for fill-finish processing with an agitation apparatus according to embodiments of the present disclosure. At 802, biomaterials are dispensed into a container. The container has a first end and a second end. At 804, the first end of the container is affixed to an agitation apparatus having: a platform rotatable about at least a first axis, at least one shaft extending from the platform, at least one arm extending from the shaft. The first end of the container is releasably affixed to the at least one arm. At 806, the container is positioned in a substantially vertical orientation. At 808, at least a portion of the container is engaged (e.g., in contact with) with the platform. At 810, the platform is moved (e.g. translated, tilted, and/or rotated) about the first axis. Moving the platform about the first axis agitates contents of the container thereby causing mixing or agitation of the contents (e.g., biomaterials). In various embodiments, the contents may be sterile. In various embodiments the contents may be a recombinant virus, monoclonal antibody, or genetically modified cell. In various embodiments, the contents may be a pharmaceutical agent. In various embodiments, the contents may be subjected to a filtering step prior to the fill-finish step.

The following Examples A-E describe exemplary drug product fill finish manufacturing processes that can be performed using the agitation platform systems disclosed herein:

### Example A

A placebo (aqueous solution) with a gelatin particle was prepared in a volume of less than or equal to 3.0 L and agitated using an agitation platform described herein. The tilt angle of the platform may be set as 5° to 10° and adjustment can be forward or reverse incremental by 1° or more intervals as required. For example, a forward incremental tilt may be set from 5° to 6°, or reverse incremental from 10° to 5°. In various embodiments, nutating mixing, i.e., the use of gentle three-dimensional (gyrating) agitation may be used. The rocking rate is set to vary between approximately 15 RPM to approximately 42 RPM. Adjustment of the rocking rate can be forward or reverse incremental by 1 RPM.

This agitation study intended to mimic an actual drug product manufacturing/filling process by using the same equipment and consumable(s) to explore the stability/steadiness of the platform over the entire filling operation. Quantity of surrogate placebo solution vs sterile product bag capacity/dimension vs product bag placement height were part of the agitation study. Solution and particles displacement information were recorded based on varying the drug product volume vs tilt angle, or drug product volume vs rocking rate, or a combination of these 3 parameters. Optimum agitation conditions, in terms of tilt angle, rocking rate and drug product volume, were obtained. The information can be used as a baseline data for related filling/manufacturing processes for pharmaceutical and/or biopharmaceutical products (e.g., gene therapy, immunotherapy), and maintaining homogeneity of the product during fill-finish processes.

### Example B

A virus-based retroviral replicating vector was prepared in a volume of less than or equal to 5.0 L and agitated using an agitation platform described herein. The resulting vials were filled to approximately 4 mL per vial. The tilt angle of the platform may be set as 5° to 10° and adjustment can be forward or reverse incremental by 5° or more. For example, a forward incremental tilt may be set from 5° to 10°, or reverse incremental from 10° to 5°. The rocking rate is set to vary between approximately 10 RPM to approximately 25 RPM. Adjustment of the rocking rate can be forward or reverse incremental by 5 RPM. This agitation study aimed to investigate the physical stability of the drug product based on manual visual inspection of the fill finish drug product vials.

Visual inspection criteria may follow those outlined in USP <788>, USP <790>, which are incorporated by reference herein with respect to visual inspection. Inherent, intrinsic and extrinsic particulates regardless of its solubility, or conformation, or configuration was subjected to a standard visual inspection guideline. Material aggregation or agglomerate phenomena was the focus of this study by varying the drug product volume vs tilt angle, or drug product volume vs rocking rate, or a combination of these 3 parameters. Settling of drug product or aggregation/agglomerate propensities was not observed. The product vials passed the defined attributes based on manual visual inspection and Acceptable Quality Limit (AQL).

### Example C

A virus-based retroviral replicating vector was prepared in a volume of less than or equal to 5.0 L and agitated using an agitation platform described herein. The resulting vials were filled to approximately 5 mL per vial. The tilt angle of the platform was set at a fixed angle of 5°. This study aimed to investigate the physical stability of the drug product based on manual visual inspection of the fill finish drug product vial. Visual inspection criteria follow those outlines in USP <788>, USP <790>, which are incorporated by reference herein with respect to visual inspection. Material aggregation or agglomerate phenomena was not observed. The product vials passed the defined attributes based on manual visual inspection and Acceptable Quality Limit (AQL). Specific numbers of vials were randomly selected for Quality Control (QC) release testing, such compendial QC testing including but not limited to the concentration profile (titer Vg/mL), pH, Osmolality, and other tests met the defined release criteria.

### Example D

A recombinant adeno-associated virus (rAAV) gene therapy drug product was prepared having a weight of approximately 800 g in solution and agitated using an agitation platform described herein. The resulting vials were filled to approximately 1.0 mL per vial. The tilt angle of the platform ranged from 5° to 10°, with a target angle of about 7°. Adjustment can be forward or reverse incremental by 1° or 2°. For example, a forward incremental tilt may be set from 5° to 6°, or reverse incremental from 7° to 5°. The rocking rate ranged from approximately 5 RPM to approximately 25 RPM, setting a target of around 20 RPM. Adjustment of the rocking rate can be forward or reverse incremental by 1 RPM. This agitation study aimed at exploring the optimum agitation conditions during the rAAV gene therapy drug product during the final fill process. Quantity of drug product versus sterile product bag capacity/dimension vs drug product placement height were part of the agitation study. The product vials passed the defined attributes based on manual visual inspection and Acceptable Quality Limit (AQL). Specified number of vials sampled (randomly selected vials) post fill finish manufacturing process met the pre-defined Release Criteria and/or Quality Attributes.

### Example E

A rAAV gene therapy drug product was prepared having a weight of approximately 600 g in solution and agitated using an agitation platform described herein. The resulting vials were filled to approximately 1.0 mL per vial. The tilt angle of the platform ranged from 5° to 10°, with a target angle of about 7°. Adjustment can be forward or reverse incremental by 1° or 2°. The rocking rate ranged from approximately 5 RPM to approximately 25 RPM, with a target of about 10 RPM. Adjustment of the rocking rate can be forward or reverse incremental by 1 RPM. This study aimed to investigate the physical stability of the drug product based on manual visual inspection of the fill finish drug product vials. Visual inspection criteria follow those outlines in USP <788>, USP <790>, which are incorporated by reference herein with respect to visual inspection. Material aggregation or agglomerate phenomena was not observed. The product vials passed the defined attributes based on manual visual inspection and Acceptable Quality Limit (AQL). Specific numbers of vials were randomly selected for Quality Control (QC) release testing, such compendial QC testing including but not limited to the concentration profile (AAV Capsid titer Vg/mL), pH, Osmolality, appearance, sub-visible particulates and other tests met the defined release criteria.

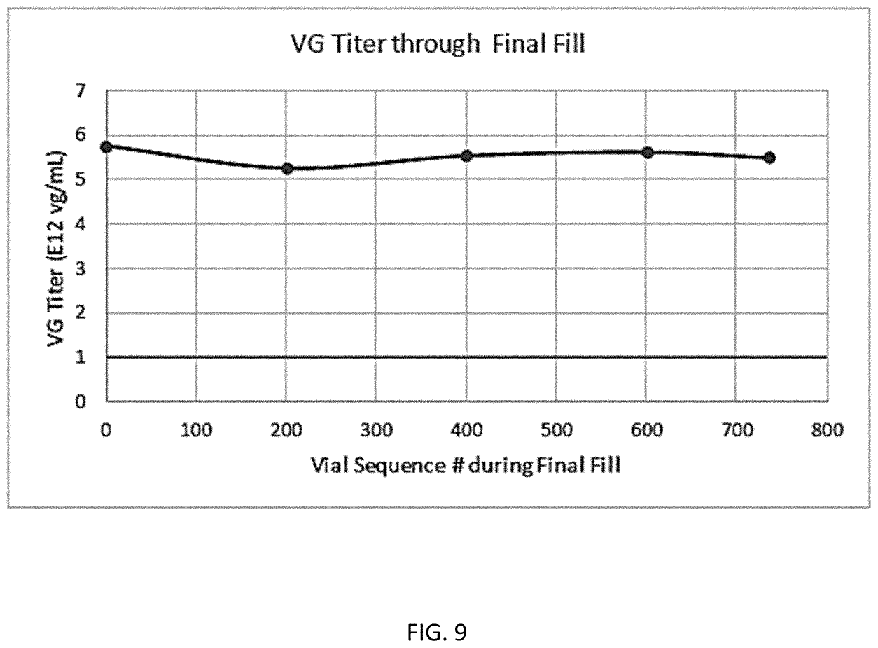

FIG. 9 shows the vector genome (Vg) titer (which is a measure of viral quantification) for vials taken during the final fill for Example E. In particular, FIG. 9 shows Vg titer through the final fill vials (top, blue line) and Vg titer data from randomly sampled vials (bottom, red line). The data shown in FIG. 9 includes a minimum Vg titer concentration requirement. The data shown in FIG. 9 indicated that the concentration profile (titer Vg/mL) is within release criteria.

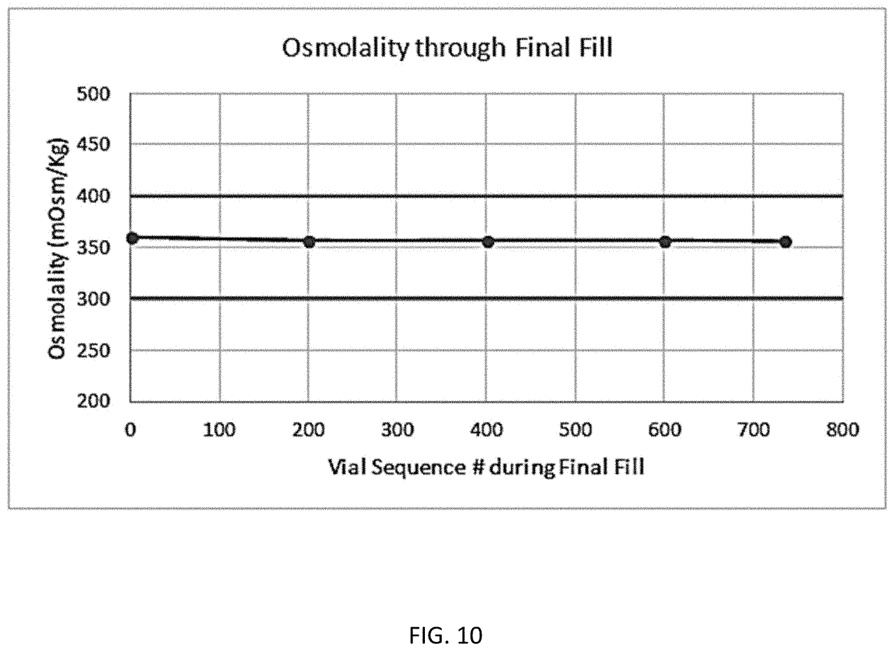

FIG. 10 shows the osmolality results for vials taken during the final fill for Example E. In particular, FIG. 10 shows osmolality through Final Fill (top and bottom, red line) for a predefined osmolality range for the drug product (middle, blue line). The data shown in FIG. 10 includes osmolality data from randomly sampled vials. The data shown in FIG. 10 indicated that the osmolality profile (mOsm/kg) is within release criteria.

In various embodiments, the vector genome concentration information, Vg titer, is a critical lot release assay for AAV vector preparations and used as a measure for dosing purposes. In various embodiments, a method for quantifying AAV vectors may include a quantitative PCR (qPCR) approach. In various embodiments, osmolality is a measure of the total number of dissolved active ion or particles in a given volume of solution given in osmol/kg. In various embodiments, osmolality measurements can be taken using an osmometer, such as Orion™ Versa Star Pro™ pH/ISE/Conductivity/Dissolved Oxygen Multi-parameter Benchtop Meter. In various embodiments, osmolality may be regularly carried out in the pharmaceutical industry, and clinical or research labs to establish the isotonicity of solutions.

In various embodiments, for both Vg titer and osmolality measurements, random vials were selected, e.g., approximately every 200th vial across the entire batch, with certain vials used for Vg titer measurements and others used for osmolality measurements. For example, vial numbers 1, 200, 300 . . . were selected for Vg titer and vial numbers 2, 201, 301 . . . were selected for osmolality). The results shown in FIGS. 9 and 10 demonstrate that Vg titer and osmolality were consistent across the different vials in the batch (i.e., ≥1 E12 Vg/mL and 300-400 mOsm/Kg), demonstrating stability of the product across the batch. In various embodiments, the Vg titer and Osmolality results shown in FIGS. 9 and 10 show that, using an approximately 7° tilt angle and 10 rpm rocking rate, the custom-made agitation platform can provide sufficient agitation for homogenous rAAV gene therapy drug product in filled vials.

The flowchart and block diagrams in the figures illustrate the architecture, functionality, and operation of possible implementations of systems, methods, and devices according to various embodiments of the present disclosure. In various alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present disclosure have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. An apparatus for aseptic fill-finish while agitating, the apparatus comprising:
an agitation device;
a platform coupled to the agitation device, the platform rotatable about a first axis;
at least one shaft extending from the platform, the shaft adjustable in length;
at least one arm extending from the shaft, the at least one arm having at least one fixation mechanism;
wherein the at least one fixation mechanism is configured to receive a container having a sealed compartment containing a biomaterial and maintain the container in a substantially vertical orientation; and
wherein the platform is configured to engage at least a portion of the container.

2. The apparatus of claim 1, wherein the shaft extends perpendicularly from a midpoint of the platform, the at least one arm extends perpendicularly from the shaft, and the at least one fixation mechanism extends perpendicularly from the at least one arm.

3. The apparatus of claim 1, wherein the platform comprises a planar surface.

4. The apparatus of claim 1, wherein the platform comprises a non-planar surface.

5. The apparatus of claim 1, wherein the at least one shaft includes a first shaft and a second shaft, the first shaft disposed proximate a first edge of the platform and the second shaft disposed proximate a second edge of the platform.

6. The apparatus of claim 5, wherein the first shaft is aligned with the second shaft.

7. The apparatus of claim 1, wherein the agitation device displaces the platform at a variable speed.

8. The apparatus of claim 1, wherein the platform is configured to rotate about a second axis.

9. The apparatus of claim 1, wherein the platform is configured to rotate about first and second axes simultaneously.

10. The apparatus of claim 1, wherein the platform is configured to move in a plane defined by the first axis and the second axis.

11. The apparatus of claim 1, wherein the container is a flexible bag.

12. The apparatus of claim 1, wherein the container comprises a polymer.

13. The apparatus of claim 1, wherein the biomaterial is selected from the group consisting of: a recombinant virus, monoclonal antibody, pharmaceutical agent, and genetically-modified cell.

14. The apparatus of claim 1, wherein the container is not fluidly coupled to a pump.

15. The apparatus of claim 1, wherein the container does not include a stirring mechanism.

16. The apparatus of claim 1, wherein the container is not fluidly coupled to a second container.

17. A method for aseptic fill-finish, the method comprising:
dispensing biomaterials into a container, the container having a first end and a second end;
affixing the first end of the container to an agitation device having:
a platform rotatable about a first axis,
at least one shaft extending from the platform,
at least one arm extending from the shaft, wherein the first end of the container is releasably affixed to the at least one arm;
positioning the container in a substantially vertical orientation;
engaging at least a portion of the second end of the container with the platform; and
rotating the platform about the first axis.

18. The method of claim 17, further comprising processing the biomaterials prior to dispensing.

19. The method of claim 17, further comprising subjecting the biomaterials to a filtration step prior to dispensing.

20. The method of claim 17, wherein the platform comprises a planar surface.

21. The method of claim 17, wherein the platform comprises a non-planar surface.

22. The method of claim 17, wherein affixing the first end of the container includes adjusting a length of the shaft.

23. The method of claim 17, wherein rotating includes displacement of the platform about a second axis.

24. The method of claim 17, wherein rotating includes displacement of the platform in a plane defined by the first axis and a perpendicular second axis.

25. The method of claim 17, wherein rotating includes displacement of the platform about the first and second axes simultaneously.

26. The method of claim 17, wherein the biomaterial is selected from the group consisting of: a recombinant virus, monoclonal antibody, pharmaceutical agent, and genetically-modified cell.

27. The method of claim 17, wherein the biomaterials are sterile.

28. The method of claim 17, wherein the container is not fluidly coupled to a pump.

29. The method of claim 17, wherein the container does not include a stirring mechanism.

30. The method of claim 17, wherein the container is not fluidly coupled to a second container.
